(19) United States
(12) Patent Application Publication
(10) Pub. No.: US 2007/0162140 A1
(43) Pub. Date: Jul. 12, 2007

(54) Method and apparatus for reconstructing a joint

(76) Inventor: Dennis M. McDevitt, Raleigh, NC (US)

Correspondence Address: Mark J. Pandiscio;Pandiscio & Pandiscio, P.C., 470 Totten Pond Road, Waltham, MA, 02451-1914, US

(21) Appl. No.: 11/638,078

(22) Filed: Dec. 13, 2006

Related U.S. Application Data
(63) Continuation-in-part of application No. 10/767,673, filed on Jan. 29, 2004, now Pat. No. 7,166,132.
Application No. 10/767,673 is a division of application No. 10/041,722, filed on Jan. 8, 2002, now Pat. No. 6,736,852.
(60) Provisional application No. 60/271,895, filed on Feb. 27, 2001.

(51) Int. Cl. A61F 2/40

(52) U.S. Cl. 623/19.14; 623/908

(57) ABSTRACT

The present invention provides a new and improved method and apparatus for reconstructing a joint. More particularly, the present invention provides a new and improved joint prothesis comprising a stem, a neck angled off the stem, and a head set at the end of the neck, wherein the stem, neck and head utilize new and improved connection mechanisms for providing a greater range of assembly configurations. Among other things, the present invention provides a new and improved joint prosthesis which permits adjustment of the longitudinal extension, lateral offset and angle of inclination of the head relative to the cut surface of the humerus. In one preferred form of the invention, there is provided a joint prosthesis assembly comprising: a stem having a proximal end and a distal end and a longitudinal axis extending therebetween; a neck extending from the proximal end of the stem at an angle to the longitudinal axis of the stem, wherein the neck comprises a proximal end and a distal end and a longitudinal axis extending therebetween, and wherein the neck further comprises a first rounded end surface and a second non-circular end surface; a trunnion for mounting the neck to the stem, wherein the trunnion comprises a recess comprising a first rounded inner surface and a second non-circular inner surface wherein: the recess is configured to selectively and snugly receive the neck therein, such that when the neck is received by the recess, the trunnion is held in a non-rotational relationship with respect to the neck's longitudinal axis while permitting rotation about the other two axes.

### REFERENCE TO PENDING PRIOR PATENT APPLICATIONS

This application:

(i) is a continuation-in-part of pending prior U.S. patent application Ser. No. 10/767,673, filed Jan. 29, 2004 by George Hadley Callaway et al. for ADJUSTABLE BONE PROSTHESES AND RELATED METHODS (Attorney's Docket No. 9417.17685-DIV), which in turn is a divisional of U.S. patent application Ser. No. 10/041,722, filed Jan. 8, 2002 by George Hadley Callaway et al. for ADJUSTABLE BONE PROSTHESES AND RELATED METHODS, which in turn claims benefit of U.S. Provisional Patent Application Ser. No. 60/271,895, filed Feb. 27, 2001 by George Hadley Callaway et al. for ADJUSTABLE HEAD PROSTHESIS FOR THE SHOULDER; and

(ii) claims benefit of pending prior U.S. Provisional Patent Application Ser. No. 60/784,684, filed Mar. 22, 2006 by Dennis M. McDevitt for METHOD AND APPARATUS FOR RECONSTRUCTING A JOINT (Attorney's Docket No. INCUMED-1 PROV).

The four above-identified patent applications are hereby incorporated herein by reference.

### FIELD OF THE INVENTION

This invention relates to surgical methods and apparatus in general, and more particularly to methods and apparatus for reconstructing a joint.

### BACKGROUND OF THE INVENTION

A shoulder joint consists of a ball-and-socket coupling of the humerus to the scapula. More particularly, the humerus forms the ball, and the glenoid cavity of the scapula forms the socket. Injury or disease to the joint often results in deterioration of the head of the humerus, leading to pain and loss of mobility and function. In such cases, it is often necessary to provide a replacement joint surface (i.e., a prosthesis) for the head of the humerus.

In general, the humeral prosthesis comprises an elongated stem for disposition in the intramedullary canal of the humerus, a neck extending from the proximal end of the stem at an angle to the longitudinal axis of the stem, and a head mounted to the neck for engagement with the glenoid cavity. In a typical procedure, the deteriorated head of the humerus is removed, the intramedullary canal of the humerus is prepared, the stem of the prosthesis is inserted down the intramedullary canal of the humerus, and then the head is secured to the proximal end of the neck.

Accurate alignment of the prosthesis is generally important for proper joint performance. In order to accommodate variations in patient anatomies, the humeral prostheis is typically provided in a range of different configurations, e.g., different size stems, different stem/neck geometries, different size heads, etc. During surgery, the surgeon selects the stem and neck configuration most appropriate for the patient, then adjusts the longitudinal position of the stem within the intramedullary canal of the humerus, and also the rotational position of the stem within the intramedullary canal of the humerus, and then selects the appropriate head, so as to achieve the best possible joint reconstruction. In general, this arrangement provides a reasonably adequate approach for properly presenting the head of the prosthesis to the glenoid cavity. However, compromises are frequently necessary due to practical inventory limitations.

Thus, it would be desirable to provide an improved approach for mounting the head on the neck of the humeral prosthesis, so as to permit the surgeon a greater range of mounting configurations in order to better accommodate variations in patient anatomies. More particularly, it would be desirable to provide the surgeon with a greater range of mounting configurations so that the prosthesis can more precisely match the 3-dimensional presentation of the humeral head to the glenoid cavity. Among other things, it would be desirable to provide a humeral prosthesis which permits adjustment of the longitudinal extension, lateral offset and angle of inclination of the head relative to the cut surface of the humerus.

In addition to the foregoing, the reconstruction of joints other than the humerus frequently use similar prosthetic arrangements. For example, with the hip joint, the femoral prosthesis typically comprises a stem for insertion into the intramedullary canal of the femur, a neck angled off the proximal end of the stem, and a head (or ball) set at the proximal end of the neck, with the head being received in a socket formed in the pelvis. For these other joint prostheses, it would also be desirable to provide an improved approach for mounting the head on the neck, so as to permit the surgeon a greater range of mounting configurations in order to better accommodate variations in patient anatomies.

### SUMMARY OF THE INVENTION

The present invention provides a new and improved method and apparatus for reconstructing a joint. More particularly, the present invention provides a new and improved joint prothesis comprising a stem, a neck angled off the stem, and a head set at the end of the neck, wherein the stem, neck and head utilize new and improved connection mechanisms for providing a greater range of assembly configurations. Among other things, the present invention provides a new and improved joint prosthesis which permits adjustment of the longitudinal extension, lateral offset and angle of inclination of the head relative to the cut surface of the humerus.

In one preferred form of the invention, there is provided a joint prosthesis assembly comprising:

a stem having a proximal end and a distal end and a longitudinal axis extending therebetween;

a neck extending from the proximal end of the stem at an angle to the longitudinal axis of the stem, wherein the neck comprises a proximal end and a distal end and a longitudinal axis extending therebetween, and wherein the neck further comprises a first rounded end surface and a second non-circular end surface;

a trunnion for mounting the neck to the stem, wherein the trunnion comprises a recess comprising a first rounded inner surface and a second non-circular inner surface wherein:
the recess is configured to selectively and snugly receive the neck therein, such that when the neck is received by the recess, the trunnion is held in a non-rotational relationship with respect to the neck's longitudinal axis while permitting rotation about the other two axes.

In another preferred form of the invention, there is provided a method for reconstructing a humeral head of a patient, the method comprising:

removing the head of the humerus;

preparing the intramedullary canal of the humerous;

providing a joint prosthesis assembly comprising:
a stem having a proximal end and a distal end and a longitudinal axis extending therebetween;
a neck extending from the proximal end of the stem at an angle to the longitudinal axis of the stem, wherein the neck comprises a proximal end and a distal end and a longitudinal axis extending therebetween, and wherein the neck further comprises a first rounded end surface and a second non-circular end surface;
a trunnion for mounting the neck to the stem, wherein the trunnion comprises a recess comprising a first rounded inner surface and a second non-circular inner surface wherein:
the recess is configured to selectively and snugly receive the neck therein, such that when the neck is received by the recess, the trunnion is held in a non-rotational relationship with respect to the neck's longitudinal axis while permitting rotation about the other two axes;

inserting the stem into the prepared intramedullary canal; and

rotating the trunnion to a desired position.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts and further wherein:

FIG. 1 is a schematic side view showing a novel humeral prosthesis, with the head spaced from the remainder of the prosthesis;

FIG. 2 is a perspective view of the humeral prosthesis shown in FIG. 1, with various components shown in exploded relation;

FIG. 3 is a perspective view like that of FIG. 2, except taken from a different angle of view;

FIG. 4 shows the underside of the trunnion component of the prosthesis;

FIG. 5 shows the underside of the head of the prosthesis;

FIGS. 6A-6D illustrate various exemplary lateral offsets which may be achieved between the stem and the head of the prosthesis;

FIG. 7 is a side view in section showing the novel humeral prosthesis of FIG. 1, but with all of the components in an assembled position;

FIG. 8 is a side view showing the head of the prosthesis angled relative to the neck;

FIG. 9 is a perspective view of the humeral prosthesis shown in FIG. 1, positioned in a humerus;

FIG. 10 is view like that of FIG. 9, except taken a different angle of view;

FIGS. 11 and 12 are schematic side views of an alternative form of the invention;

FIG. 13 is a schematic side view showing selected components from the construction shown in FIGS. 11 and 12;

FIGS. 14-16 are schematic side views illustrating various angular inclinations which may effected between the stem and the head.

### DETAILED DESCRIPTION THE PREFERRED EMBODIMENT

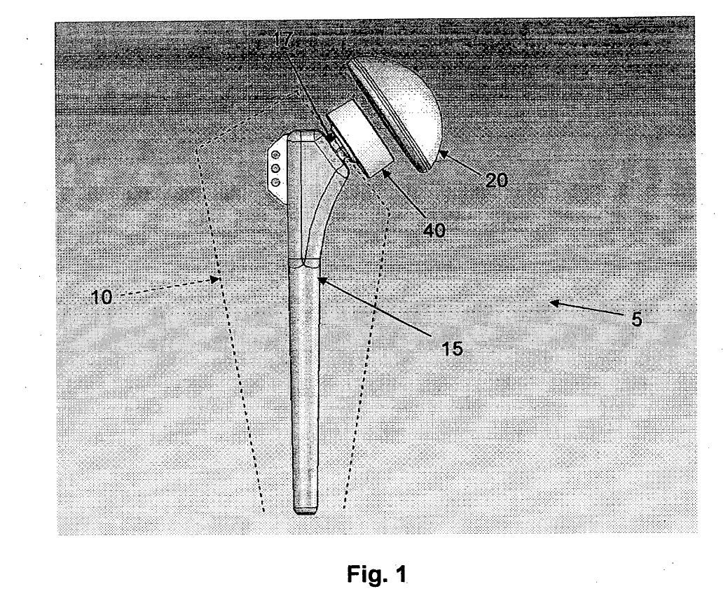

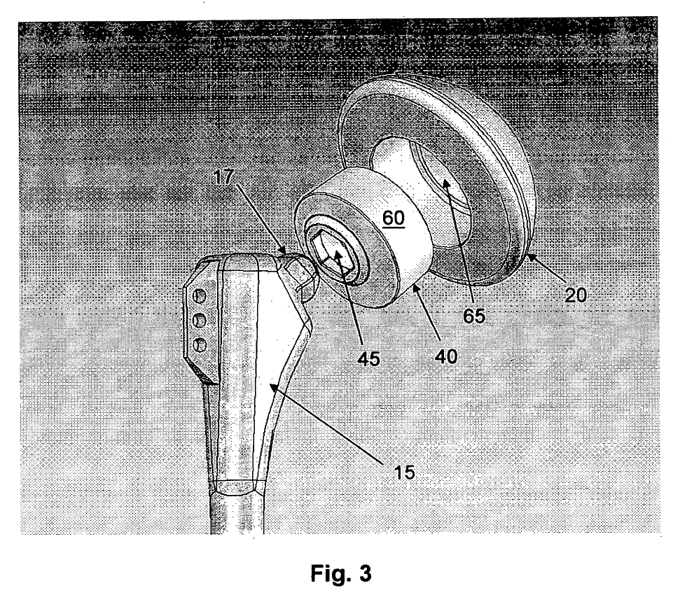

Looking first at FIGS. 1-3, there is shown a joint prosthesis 5 formed in accordance with the present invention. Prosthesis 5 is shown in a configuration suitable for use with a humerus 10, although prosthesis 5 may also be configured for use with bones of other joints, e.g., the femur of the hip joint. To the extent that prosthesis 5 may be used in joints other than that of the humeral head, the configuration of specific components may change, in ways which will be clear to those skilled in the art, according to the particular joint which is to be reconstructed.

For the purposes of clarity, joint prosthesis 5 will hereinafter be discussed in the context of the humeral head, such that prosthesis 5 will be set in humerus 10.

Humeral prosthesis 5 generally comprises a stem 15, a neck 17 and a head 20. Stem 15 is configured to be received in the intramedullary canal (not shown in FIGS. 1-3) of humerus 10, and neck 17 is angled relative to the longitudinal axis of stem 15 so as to present head 20 to the glenoid cavity of the scapula.

In accordance with one aspect of the present invention, head 20 is connected to neck 17 by means of a trunnion 40.

More particularly, neck 17 projects outwardly from stem 15 at an angle to the longitudinal axis of stem 15. Significantly, neck 17 has a non-circular cross-section. In one form of the present invention, neck 17 has a hexagonal cross-section. Alternatively, neck 17 may have another polygonal cross-section (e.g., neck 17 may have a 12-sided cross-section), or neck 17 may be ovoid.

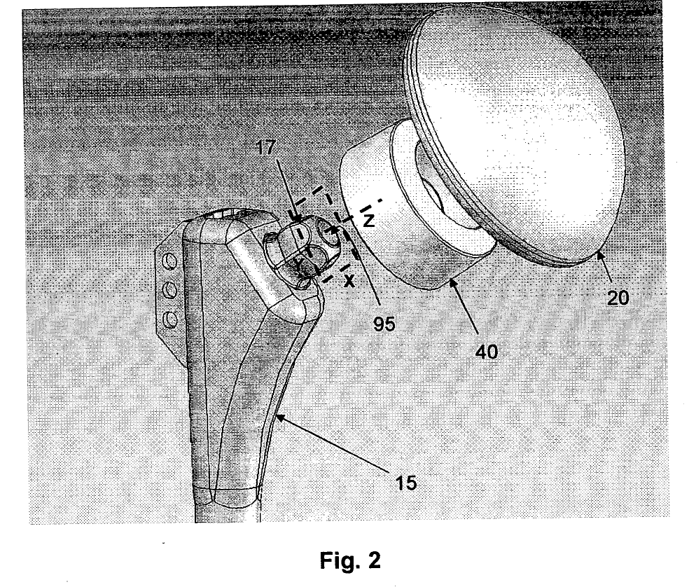

Trunnion 40 is mounted on neck 17. To this end, trunnion 40 comprises a recess 45 formed on the underside of the trunnion. Significantly, recess 45 has a non-circular cross-section which is configured to snugly receive neck 17 therein, so that trunnion 40 will be held in non-rotational relationship therewith, i.e., trunnion 40 will be prevented from rotating about the longitudinal axis of neck 17. Stated another way, and referring now to the X, Y and Z coordinates shown in FIG. 2, the non-circular cross-sections of neck 17 and recess 45 prevent trunnion 40 from rotating relative to neck 17 with respect to the Z axis, while permitting trunnion 40 to rotate relative to neck 17 with respect to the X and Y axes (the rotation about the X and Y axes may be locked through the use of a compression screw, as will hereinafter be discussed). In one form of the invention, recess 45 is formed with a cross-sectional shape with is complementary to the cross-sectional shape of neck 17, e.g., if neck 17 is formed with a hexagonal cross-section, recess 45 is formed with a like hexagonal cross-section. In another form of the invention, recess 45 is formed with a cross-sectional shape which is compatible with, but not necessarily complementary to, the cross-sectional shape of neck 17, e.g., if neck 17 is formed with a triangular cross-section, recess 45 may be formed with a hexagonal cross-section.

In any case, neck 17 and recess 45 of trunnion 40 are configured so that trunnion 40 may be held in non-rotational relation about the Z-axis while permitting rotation on neck 17 about the X and Y axes (trunnion 40 may be locked against rotation about the X and Y axes through the use of a compression screw, as will hereinafter be discussed). This is a significant aspect of the present invention and is achieved by providing neck 17 and recess 45 of trunnion 40 with non-circular, mating cross-sections.

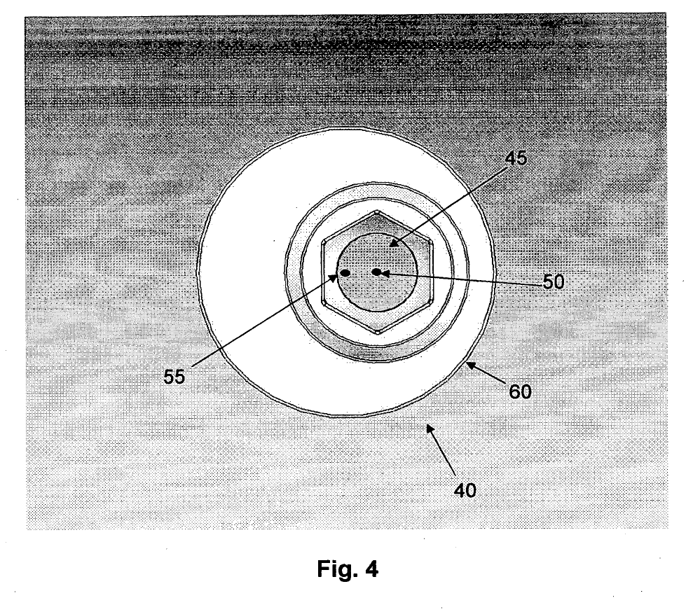

In addition to the foregoing, and looking now at FIG. 4, the longitudinal axis 50 of the trunnion's recess 45 is not co-axial with the longitudinal axis 55 of trunnion 40, i.e., the axis 50 of recess 45 is eccentric to the axis 55 of trunnion 40, for reasons which will hereafter be discussed.

The outer surface 60 of trunnion 40 is formed with a taper, in order that outer surface 60 of trunnion 40 may form one half of a Morse taper connection between trunnion 40 and head 20, as will hereinafter be discussed.

Looking now at FIGS. 1-3 and 5, head 20 is mounted on trunnion 40. To this end, head 20 comprises a recess 65 formed on the underside thereof. The inner surface 70 of recess 65 is formed with a taper, in order that inner surface 70 of recess 65 may form the other half of the Morse taper connection between trunnion 40 and head 20, as will hereinafter be discussed.

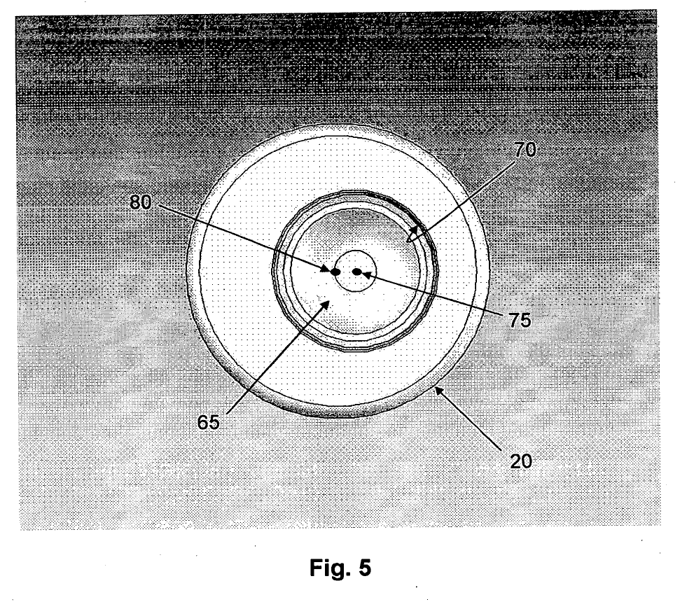

In addition to the foregoing, as seen in FIG. 5, the longitudinal axis 75 of recess 65 is not co-axial with the longitudinal axis 80 of head 20, i.e., the axis 75 of recess 65 is eccentric to the axis 80 of head 20, for reasons which will hereafter be discussed.

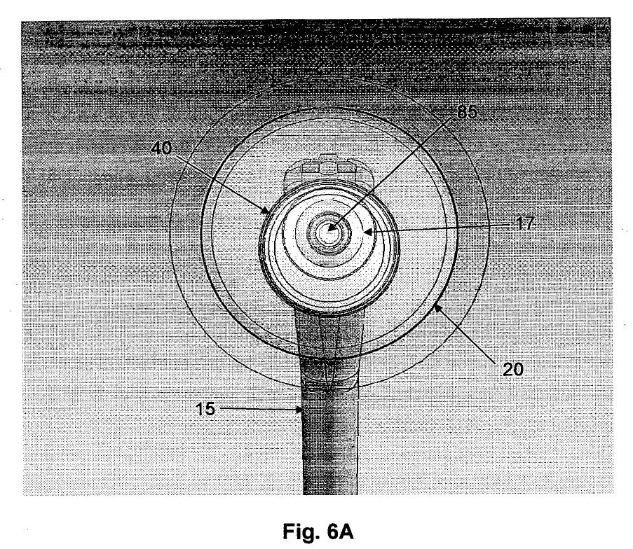

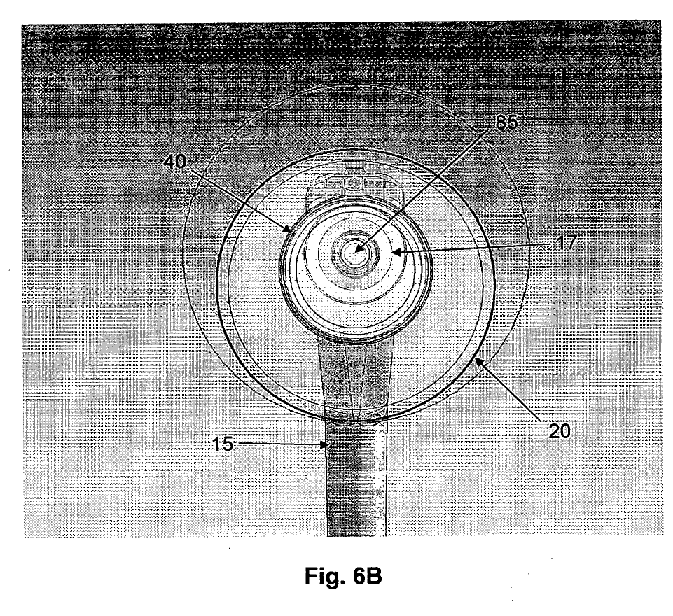

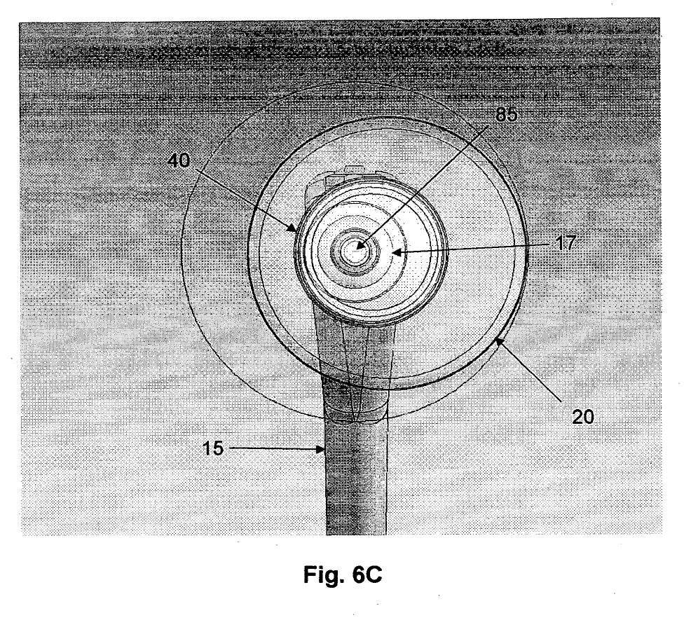

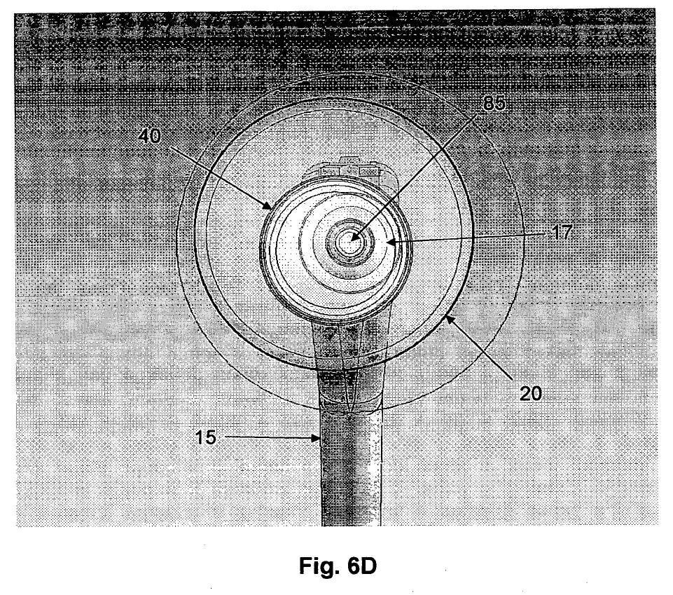

By virtue of the fact that longitudinal axis 50 of trunnion recess 45 is eccentric to longitudinal axis 55 of trunnion 40, and by virtue of the fact that longitudinal axis 75 of head recess 65 is eccentric to longitudinal axis 80 of head 20, the lateral disposition of trunnion 40 can be varied vis-à-vis the longitudinal axis of neck 17, and the lateral disposition of head 20 can be varied vis-à-vis the longitudinal axis 55 of trunnion 40, so as to permit a wide latitude in the ultimate lateral disposition of head 20 vis-à-vis the longitudinal axis of neck 17. In other words, due to the eccentric dispositions of axis 50 of trunnion recess 55 and axis 75 of head recess 65, substantial lateral offsets can be achieved between neck 17 and head 20. See, for example, FIGS. 6A-6D, which illustrate some of the possible dispositions of head 20 vis-à-vis neck 17.

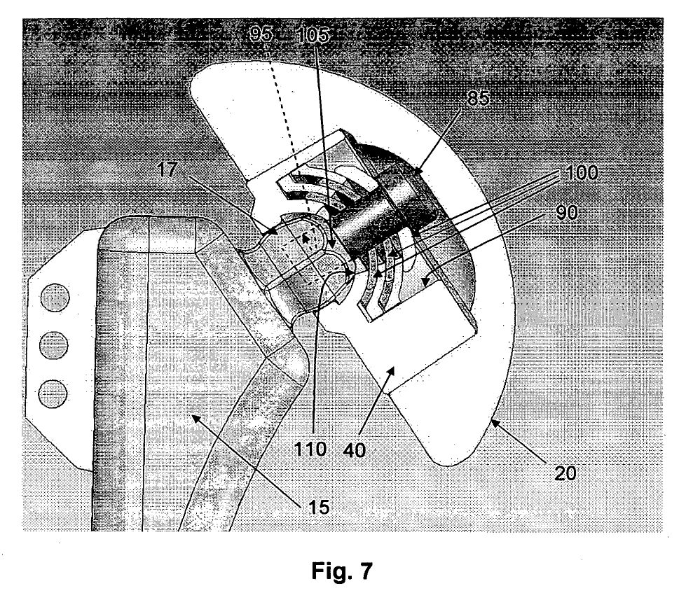

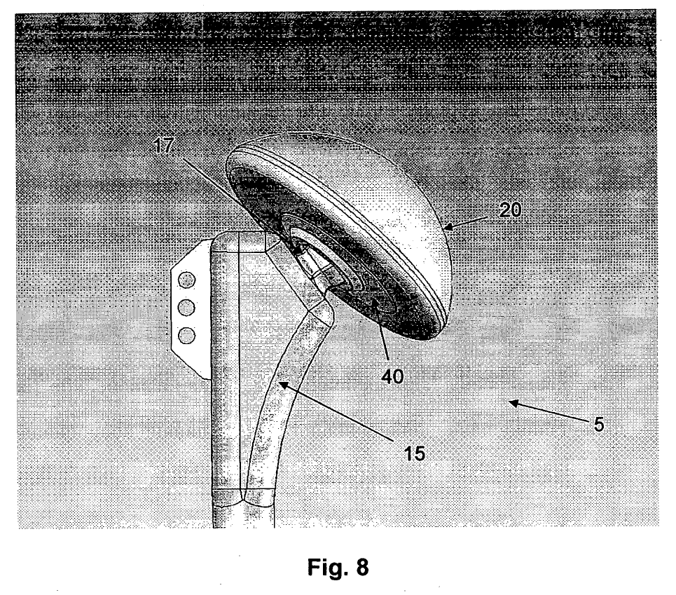

In the foregoing discussion, it was noted that trunnion 40 is mounted to neck 17 in a manner which prevents rotational movement between the parts about the Z axis. In addition, during use, it also important to stabilize the parts about the X and Y axes. To this end, in a simple form of the invention, trunnion 40 may be mounted to neck 17 with a simple press-fit or Morse taper. More preferably, however, and looking now at FIG. 7, trunnion 40 is mounted to neck 17 using a compression screw 85 which extends through a recess 90 in trunnion 40 and into a threaded bore 95 formed in neck 17. Preferably, one or more washers 100 are disposed about screw 85 so as to facilitate attachment. In one particularly preferred construction, the top end of neck 17 is rounded as shown at surface 105, and the counterpart surfaces 110 of trunnion recess 45 are correspondingly rounded, so as to permit trunnion 40 to be tilted about the X and Y axes of neck 17 before screw 85 is secured, whereby to permit head 20 to be correspondingly tilted about the X and Y axes of neck 17. See, for example, FIG. 8. In one preferred form of the invention, neck surface 105 and trunnion surface 110 have a spherical geometry. Thus, in view of the foregoing construction, trunnion 40 may be initially loosely positioned on neck 17 so that trunnion 40 may be rotated about the X and Y axes while being fixed against rotation about the Z axis; compression screw 85 may thereafter be tightened down so as to stabilize trunnion 40 vis-à-vis the X and Y axes; and head 20 may thereafter be fitted onto trunnion 40.

Humeral prosthesis 5 is preferably used as follows. First, the deteriorated head of humerus 10 is removed and the intramedullary canal of the humerus is prepared. Then trunnion 40 is loosely connected to neck 17 (i.e., with screw 85 and washers 100), and then stem 15 is inserted down the intramedullary canal of humerus 10. Next, with screw 85 set to a loosened position, trunnion 40 is pulled away from neck 17 and trunnion 40 is rotated about the Z axis to a desired position. This action serves to provide a first degree of lateral offset relative to the Z axis, due to the eccentric disposition of trunnion recess 45. Next, trunnion 40 is tilted about the X and Y axes to its desired angle of inclination. Then screw 85 is tightened down so as to lock trunnion 40 in position on neck 17. At this point, a first degree of lateral offset has been achieved, and the angle of inclination has been obtained. Finally, head 20 is oriented as desired and then press-fit onto trunnion 40, with the Morse taper securing head 20 in position. As head 20 is oriented as desired, a second degree of lateral offset is achieved, due to the eccentric disposition of head recess 65.

It will be appreciated that, by virtue of the eccentric dispositions of axis 50 of trunnion recess 45 and axis 75 of head recess 65, head 20 can be given a desired lateral disposition relative to the axis of neck 17. At the same time, by giving neck 17 and trunnion recess 45 non-circular, mating cross-sections, trunnion 40 will be held in non-rotational relation on neck 17. Furthermore, due to the provision of rounded neck surface 105 and rounded trunnion surface 110, trunnion 40 (and ultimately head 20) can be given a desired angular disposition.

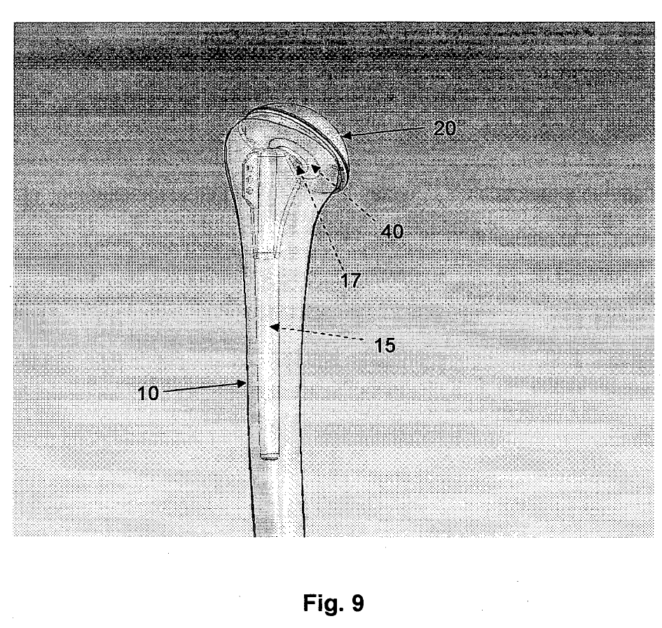

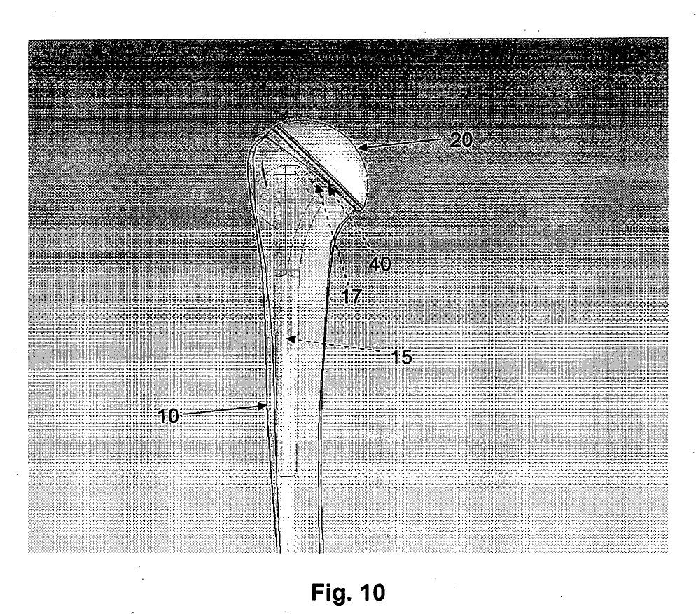

FIGS. 9 and 10 show humeral prosthesis 5 fully inserted in humerus 10, with head 20 being presented with a preferred lateral and angular disposition.

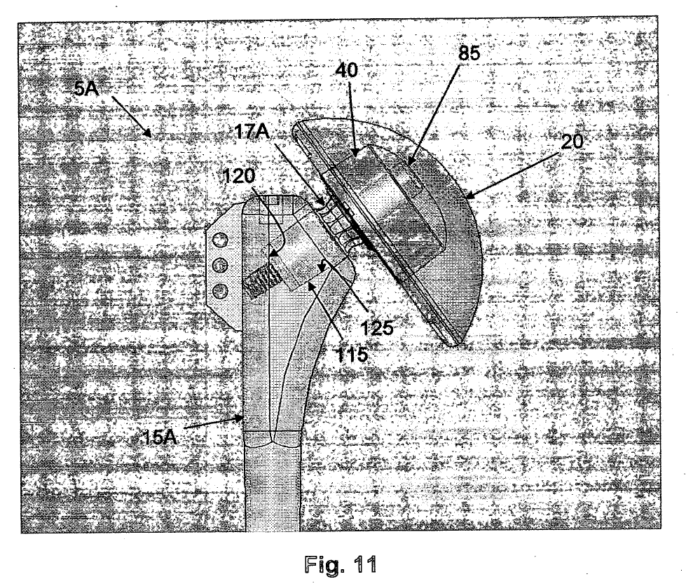

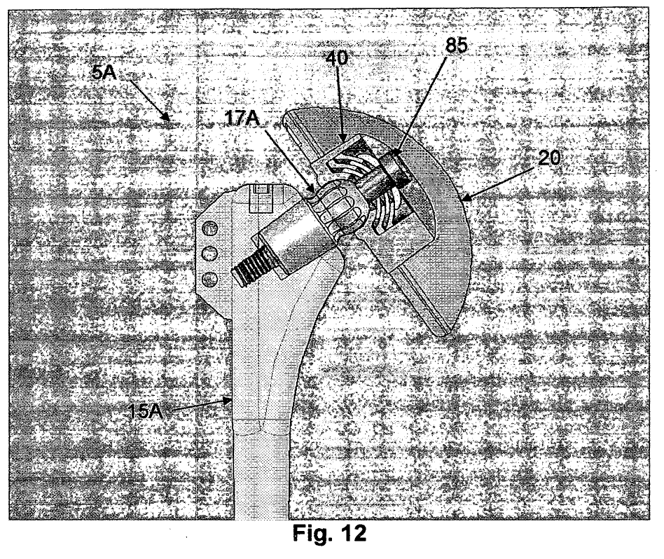

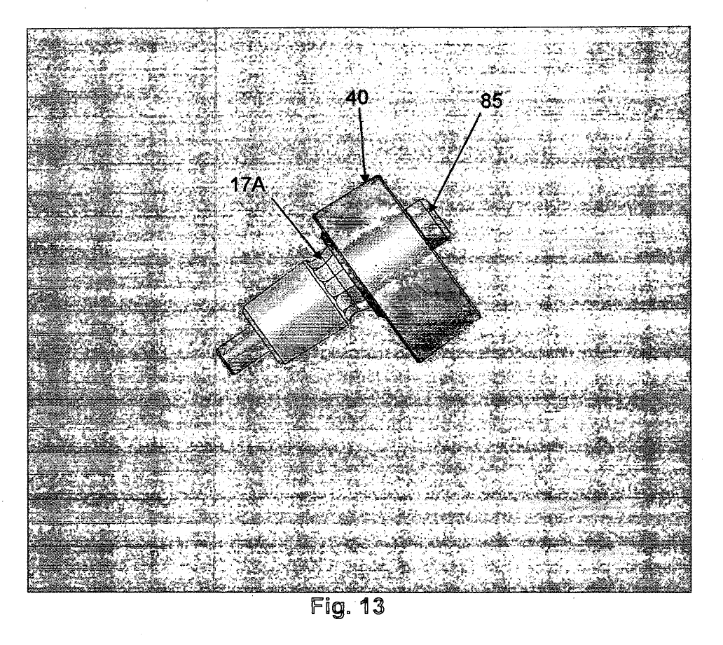

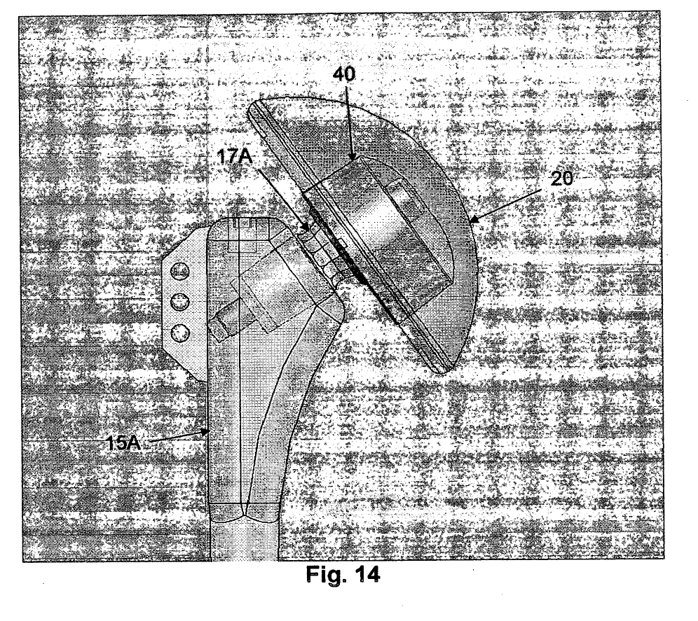

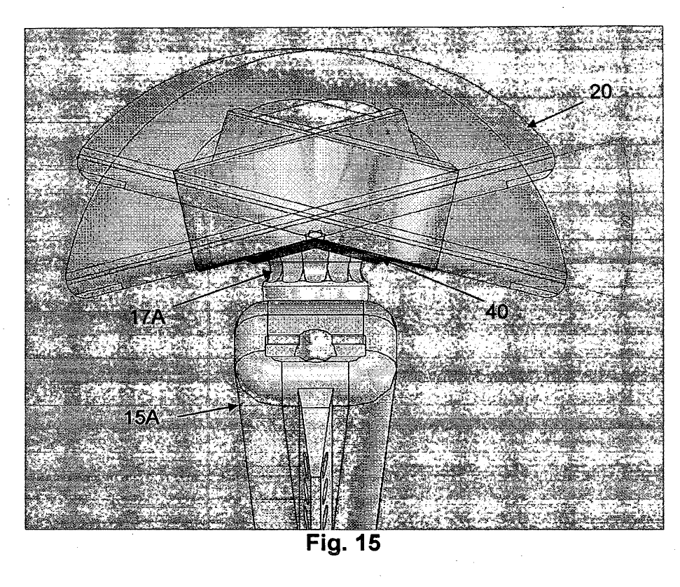

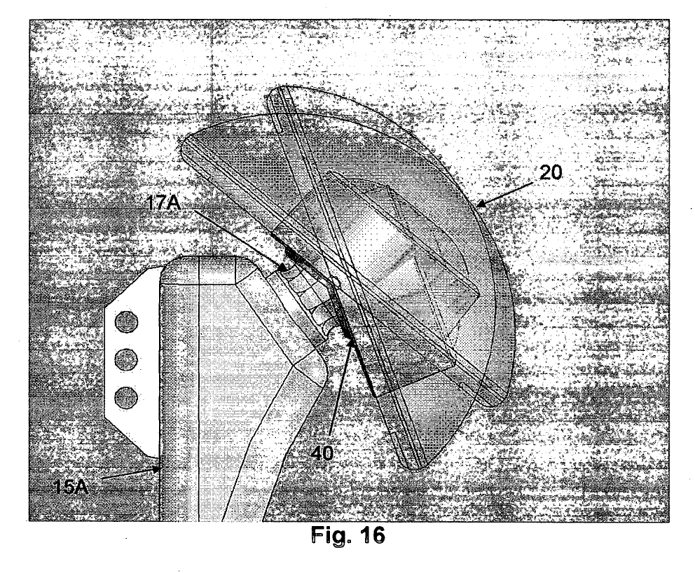

In some circumstances, it may be desirable to vary the length of neck 17 so as to adjust the disposition of head 20 relative to stem 15 (and hence humerus 10). To this end, and looking now at FIGS. 11-13, there is shown a humeral prosthesis 5A which is substantially the same as humeral prosthesis 5 discussed above, except to the extent hereinafter discussed. More particularly, humeral prosthesis 5A utilizes a modular neck 17A which mates to a stem 15A. Modular neck 17A is similar, on its distal end, to neck 17. Modular neck 17A includes a tapered surface 115 on its proximal end. Stem 15A includes a recess 120 having tapered side wall 125. The modular neck's tapered surface 115 and the stem's side wall 125 together form the two halves of a Morse taper. As a result of this construction, the longitudinal offset of head 20 relative to stem 15A can be varied by selecting a modular neck 17A of appropriate length. Thus, with this form of the invention, it is possible for the surgeon to adjust, relative to the cut surface of the humerus, (i) the longitudinal offset of head 20, (ii) the lateral offset of head 20, and (iii) the angle of inclination of head 20. See also FIGS. 14-16.

### MODIFICATIONS

It will be appreciated that still further embodiments of the present invention will be apparent to those skilled in the art in view of the present disclosure. It is to be understood that the present invention is by no means limited to the particular constructions herein disclosed and/or shown in the drawings, but also comprises any modifications or equivalents within the scope of the invention.

What is claimed is:

1. A joint prosthesis assembly comprising:
a stem having a proximal end and a distal end and a longitudinal axis extending therebetween;
a neck extending from the proximal end of the stem at an angle to the longitudinal axis of the stem, wherein the neck comprises a proximal end and a distal end and a longitudinal axis extending therebetween, and wherein the neck further comprises a first rounded end surface and a second non-circular end surface;
a trunnion for mounting the neck to the stem, wherein the trunnion comprises a recess comprising a first rounded inner surface and a second non-circular inner surface wherein:
the recess is configured to selectively and snugly receive the neck therein, such that when the neck is received by the recess, the trunnion is held in a non-rotational relationship with respect to the neck's longitudinal axis while permitting rotation about the other two axes.

2. An assembly according to claim 1 wherein the second non-circular end surface of the neck is polygonal.

3. An assembly according to claim 1 wherein the second non-circular end surface of the neck is hexagonal.

4. An assembly according to claim 1 wherein the second non-circular end surface of the neck is ovoid.

5. An assembly according to claim 1 wherein the second non-circular end surface of the neck is complementary to the second non-circular inner surface of the recess.

6. An assembly according to claim 5 wherein the second non-circular end surface of the neck is hexagonal and the second non-circular inner surface of the recess is hexagonal.

7. An assembly according to claim 1 wherein the neck comprises a second non-circular end surface which is compatible with the second non-circular inner surface of the recess.

8. An assembly according to claim 7 wherein the second non-circular end surface of the neck is triangular and the second non-circular inner surface of the recess is hexagonal.

9. An assembly according to claim 1 wherein the assembly further comprises a head for selective attachment to the proximal end of the trunnion, wherein the head comprises a proximal end, a distal end and a longitudinal axis extending therebetween.

10. An assembly according to claim 9 wherein the proximal end of the trunnion comprises a taper.

11. An assembly according to claim 9 wherein the distal end of the head comprises a head recess, wherein the head recess extends along a longitudinal axis.

12. An assembly according to claim 11 wherein the recess of the head comprises a taper.

13. An assembly according to claim 9 wherein, when the trunnion and the head are attached to one another, the proximal end of the trunnion comprises one half of a Morse taper connection and the distal end of the head comprises the other half of a Morse taper connection.

14. An assembly according to claim 9 wherein the longitudinal axis of the head recess is not co-axial with the longitudinal axis of the head.

15. An assembly according to claim 1 wherein the assembly further comprises a screw for securing the trunnion to the neck so as to prevent rotation about said other two axes.

16. An assembly according to claim 1 wherein the neck is detachable from the stem.

17. A method for reconstructing a humeral head of a patient, the method comprising:
removing the head of the humerus;
preparing the intramedullary canal of the humerous;
providing a joint prosthesis assembly comprising:
a stem having a proximal end and a distal end and a longitudinal axis extending therebetween;
a neck extending from the proximal end of the stem at an angle to the longitudinal axis of the stem, wherein the neck comprises a proximal end and a distal end and a longitudinal axis extending therebetween, and wherein the neck further comprises a first rounded end surface and a second non-circular end surface;
a trunnion for mounting the neck to the stem, wherein the trunnion comprises a recess comprising a first rounded inner surface and a second non-circular inner surface wherein:
the recess is configured to selectively and snugly receive the neck therein, such that when the neck is received by the recess, the trunnion is held in a non-rotational relationship with respect to the neck's longitudinal axis while permitting rotation about the other two axes;
inserting the stem into the prepared intramedullary canal; and
rotating the trunnion to a desired position.

18. A method according to claim 17 wherein the assembly further comprises a head for selective attachment to the proximal end of the trunnion, and wherein the method further comprises attaching the head to the proximal end of the trunnion.

19. A method according to claim 18 wherein the head comprises a longitudinal axis and a head recess comprising a longitudinal axis, and further wherein the longitudinal axis of the head recess is not co-axial with the longitudinal axis of the head.

20. A method according to claim 19 wherein the method further comprises rotating the head about the trunnion to a desired position.

21. A method according to claim 17 wherein the assembly further comprises a screw for selectively securing the trunnion to the neck.

22. A method according to claim 17 wherein the step of providing a joint prosthesis includes attaching the neck to the stem.

23. A method according to claim 22 wherein the method further comprises tilting the trunnion about the longitudinal axis of the neck to a desired tilted position.
